(19) United States
(12) Patent Application Publication
(10) Pub. No.: US 2022/0168591 A1
(43) Pub. Date: Jun. 2, 2022

(54) COCHLEAR IMPLANT AND METHOD OF GENERATING STIMULATIONS FOR A COCHLEAR IMPLANT

(71) Applicants: Nurotone Medical Ltd., Rosh Ha'Ayin (IL); NORTHWESTERN UNIVERSITY, Evanston, IL (US)

(72) Inventors: Dan David ALBECK, Givat Shmuel (IL); Claus Peter RICHTER, Skokie, IL (US); Yingyue XU, Chicago, IL (US); Xiaodong TAN, Wilmette, IL (US); Natan DAVIDOVICS, Rehovot (IL)

(73) Assignees: Nurotone Medical Ltd., Rosh Ha'Ayin (IL); NORTHWESTERN UNIVERSITY, Evanston, IL (US)

(21) Appl. No.: 17/427,191

(22) PCT Filed: Feb. 6, 2020
(86) PCT No.: PCT/IL2020/050146

§ 371 Date: Jul. 30, 2021

Related U.S. Application Data
(60) Provisional application No. 62/801,771, filed on Feb. 6, 2019.

(51) Int. Cl. A61N 5/06; G02B 6/42

(52) U.S. Cl. CPC A61N 5/0622; A61N 5/0603; G02B 6/4203; A61N 2005/0626; A61N 2005/0659; A61N 2005/063; A61N 2005/0651; A61N 2005/0605

(57) ABSTRACT

Disclosed is a cochlear implant (Cl) and a method of generating stimulations for the CI. The Cl may include, at least one optical signal generator configured to generate a plurality of optical signals having a wavelength of at most 1600 nm, and a plurality of light emitters, for delivering the optical signals to different locations along a cochlear nerve. The method may further include optimizing the stimulation energy through selection of stimulation wavelengths and stimulation pulse shapes.

### GOVERNMENT INTEREST STATEMENT

This invention was made with US government support under Grants No. R01-DC011855 and R56DC017492 awarded by the National Institute on Deafness and Other Communication Disorders, part of the National Institutes of Health, USA. The US government has certain rights in the invention.

### FIELD OF THE INVENTION

The present invention relates to neural stimulation in neuroprostheses, in general and particularly in cochlear implants. More particularly, the present invention relates to systems and methods for generating optical neural stimulations for a cochlear implant.

### BACKGROUND OF THE INVENTION

A cochlear implant (CI) is a surgically implanted neuroprosthetic device that provides a sense of sound to a person with severe-to-profound sensorineural hearing loss. Commercially available CIs include an array of electrical contacts (CI electrode), which is surgically inserted into scala tympani of the cochlea to stimulate the cochlear nerve. Most commercially available CIs include 12-22 electrical contacts. The contact location along the cochlear spiral is selected in order to stimulate a different part of the cochlear nerve, which corresponds to a different acoustic frequency band. The CI also includes a microphone configured to capture an acoustical signal (e.g., speech, music and the like) and a sound processor that is configured to divide the acoustical signal into a number of frequency bands corresponding to the number of electrical contacts of the CI electrode placed along the cochlear spiral. The processor than determines to which electrical contact and at which intensity an electrical current should be delivered in order to stimulate the corresponding location along the cochlear nerve.

Electrical properties of the cochlear tissue result in a wide current spread with a corresponding wide section of the cochlear nerve being stimulated by each of the electrical contacts, resulting in up to 8 independent frequency bands along the cochlea.

Some improvements were made to increase the spatial resolution of the electrical stimulation through peri-modiolar electrode designs which position the electrode and its electrical contacts closer to the cochlear neurons, or multipolar stimulation, which uses more than one of the electrode contacts to narrow the current field, or current steering that uses interaction and interference between neighboring channels to evoke a percept, which is between the sites of the electrode contacts used to deliver the current. Current steering increases the number of pitches a CI user can perceive.

Neural stimulation with photons has been proposed for a next generation of neural prostheses including CIs. The potential benefit of photonic stimulation is its spatially selective activation of small neuron populations. Stimulating smaller spiral ganglion neuron (SGN) populations along the cochlea provides a larger number of independent channels to encode the acoustic information. Hearing could be restored at a higher fidelity and performance in noisy listening environments, performance using tonal languages, and music appreciation is likely to improve. The required energy to stimulate nerves depends on the stimulation technology. Electrical current is the most efficient one, while optical stimulation requires higher stimulation energies and additionally the limited efficiency of converting electrical into radiant energy (wall-plug-efficiency) further to stimulation light increases the consuming energy and the heat dissipation.

There are two known methods for optical neural stimulation of the cochlea, optogenetics and infrared neural stimulation (INS). Optogenetics requires a viral vector to express photosensitive ion channels in the membrane of the target neurons. INS does not require such treatment because during INS the fluid in the target tissue absorbs the photons and their energy is converted into heat. The result is a rapid, spatially confined heating (dT/dt) that leads to capacitive changes of the cell membrane resulting in a depolarizing current, activation of temperature sensitive ion channels, such as transient receptor potential channels, changes in gating dynamics of potassium and sodium channels, modulations of GABAergic transmission (e.g., pertaining to or affecting the neurotransmitter GABA (Gamma-Aminobutyric Acid), activation of a second messenger, calcium release in the cell, or to mechanical events such as stress relaxation waves with measurable pressure.

The wavelengths previously used for neural stimulation are around 1900 nm as these wavelengths have a high electro-magnetic (EM) radiation absorption coefficient in the fluid of the target tissue (e.g., saline solution, water, etc.), as illustrated in FIG. 1. FIG. 1 illustrates the absorption coefficient of the radiant energy in water as a function of wavelengths at the optical spectrum between 1200 nm-2400 nm.

In addition to the high radiation absorption coefficient, wavelengths around 1900 nm were also selected due to their low scattering coefficient, and clinical lasers like Ho:YAG and Thulium lasers that were available at this wavelengths range.

In optical cochlear implants, the stimulating element is inserted into the scala tympani to stimulate the spiral ganglions (SG) of the cochlear nerve that are located in the Rosenthal's canal. The radiant energy that is delivered towards the SG is absorbed and scattered by all the tissues located in the pathway of the photons from the scala tympani to the SG and the auditory nerve, including the modiolar bone. The tissue in the path of photons beam scatters the delivered photons and can drastically increase the spot size and reduce the radiant energy per unit area at the target tissue. The scattering results in loss of the spatial selectivity of the stimulation and further increases the required radiant energy for the stimulation. Accordingly, based on previous experiments conducted using EM energy having wavelength between 1850-2100 nm that showed a high safety range between radiant energy required for stimulation and the tissue ablation energy (e.g., that causes damage), it was concluded that shorter wavelengths are less or non-suitable for cochlear neural stimulation.

The decrease of radiant energy through tissue is described by the extinction coefficient (e), which is the sum of the loss through scattering (s) and absorption (wa) of the photons. While high scattering of the photons is dominant at shorter wavelengths, high absorption dominates at longer wavelengths. It is not surprising that it has been argued that shorter wavelengths are less beneficial for INS of the cochlear nerve due to the scattering of the photons, which results in broadening of the beam spot size and loss of spatial selectivity of the stimulation.

Radiation wavelengths between 1250 nm and 1600 nm are commonly used in the telecommunications industry and low-cost and readily available laser and light delivery technologies have already been developed. Furthermore, the wall-plug-efficiency of IR laser diodes is typically larger for shorter wavelengths.

Therefore, it would be beneficial if a device and method can be found that uses shorter wavelengths in the cochlea with the possibility to use the low cost and readily available technology already developed for the telecommunications industry.

### SUMMARY OF THE INVENTION

Some aspects of the invention may be directed to a cochlear implant (CI), comprising: at least one optical signal generator configured to generate a plurality of optical signals having a wavelength of at most 1600 nm; and a plurality of light emitters, for delivering the optical signals to different locations along a cochlear nerve.

In some embodiments, at least one optical signal generator may be configured to generate the signals at a wavelength of 1300-1460 nm. In some embodiments, the plurality of light emitters are optical projection element selected from: optical gratings, lenses, mirrors and prisms and the cochlear implant further comprising at least one waveguide for delivering the generated optical signals from the at least one optical signal generator to the plurality of light emitters. In some embodiments, the at least one waveguide is made from optical polymeric material which is shaped to fit a cochlea. In some embodiments, each waveguide may be shaped to fit a cochlea of a specific patient.

In some embodiments, the CI may include a bundle of waveguides and wherein the bundle of waveguides is shaped to fit a cochlea of a specific patient. In some embodiments, the at least one waveguide may include one or more optical amplifiers, embedded in the at least one waveguide for amplifying at least some of the plurality of optical signals. In some embodiments, the at least one optical signal generator is a photon generating source selected from, laser diode and light emitting diodes (LEDs).

In some embodiments, the optical signal generator may be an electrical power source and the plurality of light emitters are selected from: laser diodes and LEDs and the cochlear implant further comprising at least two wires configured to provide electricity to the laser diodes or the LEDs.

In some embodiments, the CI may further include: a receiver configured to receive instructions from a controller. In some embodiments, the controller may be configured to: control the at least one optical signal generator to generate the plurality of optical signals. In some embodiments, controller may further be configured to: control the at least one optical signal generator to generate the plurality of optical signals at a selected pulse shape, selected from: a square shaped pulse, a ramp up shaped pulse, a ramp down shaped pulse, a triangular shaped pulse and exponentially rising pulse. In some embodiments, the controller may further be configured to: control the at least one optical signal generator to generate the plurality of optical signals at two or more different wavelengths.

In some embodiments, the CI may further include: a first optical signal generator configured to generate optical signals at a first wavelength; and a second optical signal generator configured to generate optical signals at a second wavelength. In some embodiments, the controller may further be configured to: control the first optical signal generator to generate a first portion of the plurality of optical signals; and control the second optical signal generator to generate a second portion of the plurality of optical signals.

In some embodiments, the controller may further be configured to: receive a captured acoustical signal; divide the acoustical signal into a plurality of frequency bands; assign each frequency band with a specific light emitter; and control the at least one optical signal generator to deliver at least one optical signal to each one of the assigned light emitters according the acoustical signal.

Some aspects of the invention may be directed to a method of generating stimulations for a cochlear implant, comprising: generating, by an optical signal generator, a plurality of optical signals having a wavelength of at most 1600 nm; and delivering the generated plurality of optical signals at the one or more locations in a cochlea using one or more light emitters.

In some embodiments, a wavelength of the generated optical signals may be at a range of 1300-1460 nm. In some embodiments, the optical stimulations may be generated at a selected pulse shape, selected from: a square shaped pulse, a ramp up shaped pulse, a ramp down shaped pulse, a triangular shaped pulse and exponentially rising pulse. In some embodiments, generating the plurality of optical stimulations is in two different wavelengths. In some embodiments, a first wavelength is selected to penetrate to a first tissue penetration depth and the second wavelength is selected to penetrate to a second tissue penetration depth, deeper than the first tissue penetration depth.

In some embodiments, the method may further include amplifying at least some of the plurality of optical signals using one or more optical amplifiers, embedded in at least one waveguide for delivering the plurality of optical signals to the one or more light emitters. In some embodiments, the method may further include: capturing an acoustical signal; dividing the acoustical signal into a plurality of frequency bands; assigning each frequency band with a specific light emitter; and delivering at least one optical signals to each one of the assigned light emitters according to an acoustical signal.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 presents a graph showing the absorption coefficient of the radiant energy in water at wavelengths between 1200 nm-2400 nm as known in the art;

FIG. 2A shows a block diagram of a CI device, according to some embodiments of the invention;

FIG. 2B shows an illustration of an implant according to some embodiments of the invention;

FIG. 3. is a flowchart of a method of generating stimulations by a CI device according to some embodiments of the invention;

FIG. 4 shows graphs of compound action potential (CAP) during optical stimulation at various wavelengths according to some embodiments of the invention;

FIGS. 5A-5C shows graphs of the nerve response to optical stimulation. CAP values vs. the stimulation pulse energy, when the pulses are provided at 3 different wavelengths and various pulse shapes, according to some embodiments of the invention; and

FIG. 6 shows graphs of the CAP and the radiant energy for stimulation threshold at 3 different wavelengths and various pulse shapes relative to these values at 1860 nm and square pulse shape, according to some embodiments of the invention

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

Some aspects of the invention may be related to a CI and a method of generating stimulations for a CI that utilizes optical stimulations. A CI and method according to some embodiments of the invention may generate light (e.g., laser or LED) pulses having a wavelength shorter than 1600 nm, for example, between 1460-1300 nm

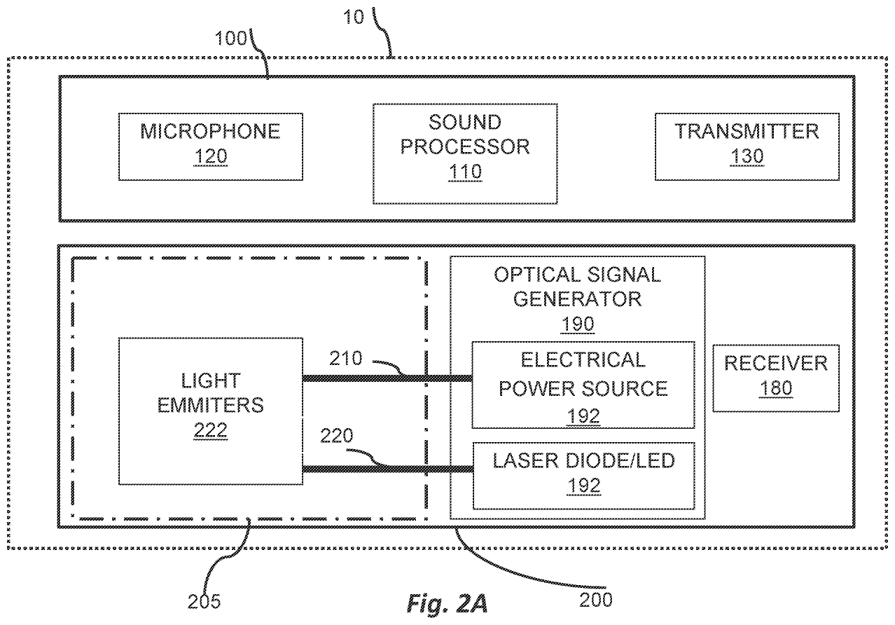

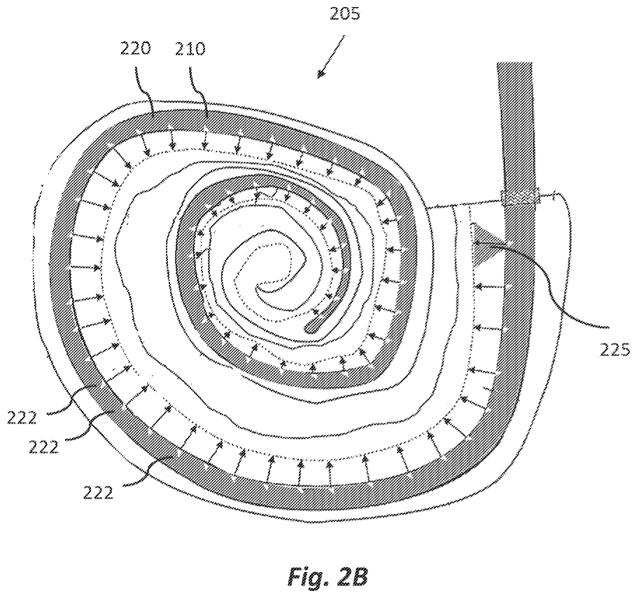

Reference is made to FIG. 2A, which shows a block diagram of an exemplary device 10 (e.g., an IC device) according to some embodiments of the invention. Device 10 may include a controller 100 and an implant 200. Controller 100 may include: a sound processor 110, a microphone 120 and a transmitter 130. Controller 100 may be a standalone devise not physically connected to implant 200. Sound processor 110 may be a central processing unit (CPU), a chip or any suitable computing or computational device. Sound processor 110 may be configured to process acoustical signals, captured by microphone 120, encode them and transmit the encoded signals to implant 200 to generate optical signals to be delivered to stimulate the cochlear nerve. Sound processor 110 may include an operating system, a memory and an executable code. Sound processor 110 may be configured to carry out methods described herein, for example, methods of generating stimulations by a CI. Microphone 120 may be any suitable device known in the art configured to capture acoustic singles, for example, speech, music, singing and the like. Transmitter 130 may be any device that may be configured to transmit instructions and/or encoded signals (e.g., wirelessly using any known method) to a receiver 180 included in implant 200.

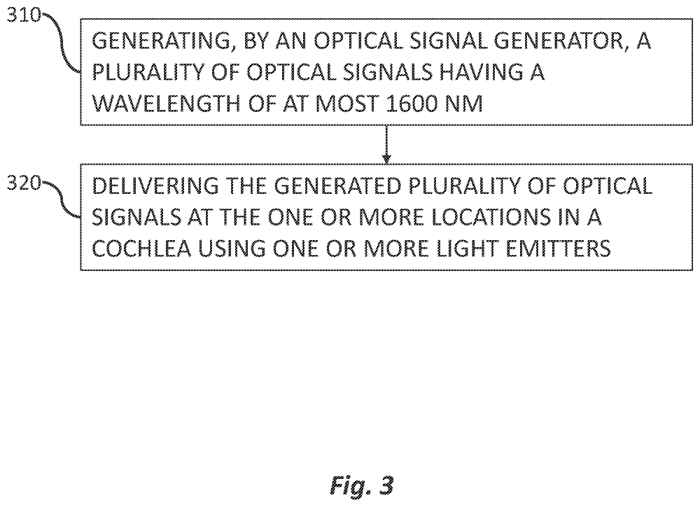

Implant 200 may include: a receiver 180 configured to receive encoded signals from sound processor 110 via transmitter 130 and to transmit them to an optical signal generator 190. In some embodiments, optical signal generator 190 may be configured to generate optical signals and deliver the optical signals to a plurality of light emitters 222 included in an optrode 220, illustrated and discussed in detail with respect to FIG. 2B. In some embodiments, optical signal generator 190 may be configured to generate a plurality of optical signals having a wavelength of at most 1600 nm, for example, at wavelengths of 1300-1460 nm, as discussed herein below with respect to the method of FIG. 3 and FIGS. 4-7. In some embodiments, optical signal generator 190 may provide signals to at least some light emitters 222 according to instructions received from sound processor 110.

In some embodiments, optical signal generator 190 may be an electrical power source 192 configured to provide electrical signals via a plurality of wires to light emitters 222 that may each include one or more photons generating elements, such as, LEDs, laser diodes and the like. In some embodiments, optical signal generator 190 may be a photons generating source 194, such as laser diode and/or LEDs, configured to provide light (e.g., photons) via one or more waveguides (e.g., optical fibers and/or light guides) to light emitters 222 that may each include optical projection elements, such as optical gratings, lenses, mirrors, prisms and the like. In some embodiments, optical generator 190 may provide light via at least one of the waveguides according to encoded signals received from sound processor 110.

Reference is now made to FIG. 2B which is an illustration of an implant 200 according to some embodiments of the invention. Implant 200 may include an optrode 205 and a plurality of light emitters 222. In some embodiments, the optrode 205 may be adapted to be implanted inside the cochlea such that light emitters 222 may be located along the cochlea or along the auditory nerve. As should be understood by one skilled in the art, the 50 light emitters illustrated in FIG. 2B are given as an example only, and the invention is not limited to any specific number of light emitters.

Optrode 205 may include one or more waveguides 220 for delivering photons generated by photon generating sources 194 to light emitters 222, when light emitters 222 are optical projection elements, such as optical gratings, lenses, mirrors, prisms and the like. Alternatively, optrode 205 may include one or more electrical wires 210 for delivering electricity from electrical power source 192, when light emitters 222 are photon generating elements, such as LEDs, laser diodes, and the like.

In some embodiments, when optrode 205 includes one or more waveguides, each may be made from optical polymeric materials that has high transmission and low scattering of the used wavelength, for example, polyimides acrylates and the like.

In some embodiments, one or more waveguides may be designed and shaped to fit a cochlea. In some embodiments, each waveguide is designed and shaped to fit a cochlea of a specific patient. For example, a three-dimension (3D) model of the cochlea of the specific patient may be generated from MRI or CT images of the cochlea. A 3D waveguide may be manufactured or printed to fit this specific cochlea 3D structure.

In some embodiments, other materials can be used to form the waveguide, for example, glass.

In some embodiments, the one or more waveguides may each include one or more optical amplifiers, embedded in each waveguide for amplifying at least some of the plurality of optical signals. Optical amplifier can be produced by doping of glass fibers or embedding die materials in polymer fibers or any other method known in the art.

In some embodiments, light emitters 222 may be configured to provide optical stimulation to a location along the cochlear nerve or along any other auditory nerve. The light illuminated by each light source may spread over area 225 (illustrated in dark grey).

Reference is now made to FIG. 3, which is a flowchart of generating stimulations for a cochlear implant according to some embodiments of the invention. The method of FIG. 3 may be performed by CI 10. In step 310, a plurality of optical signals having a wavelength of at most 1600 nm may be generated by an optical signal generator. For example, optical signal generator 190 may include a photon source 194, thus may generate pulses of photons (e.g., light) via at least one waveguide. In yet another example, pulses of electrical current may be provided from an electrical power source 192.

In step 320, the generated plurality of optical signals may be delivered at the one or more locations in a cochlea using one or more light emitters (e.g., light emitters 222). For example, optical projection element, such as, optical gratings, lenses, mirrors, prisms and the like may deliver pulses of photons generated by photons source 194. In yet another example, photon generating elements, such as LEDs, laser diodes and the like powered by electrical pulses generated by power source 192, may generate and deliver the photon pulses in situ, near the nerve.

In some embodiments, at least some of the plurality of optical signals may be amplified using one or more optical amplifiers, embedded in at least one waveguide for delivering the plurality of optical signals to the one or more light emitters, as discussed herein above.

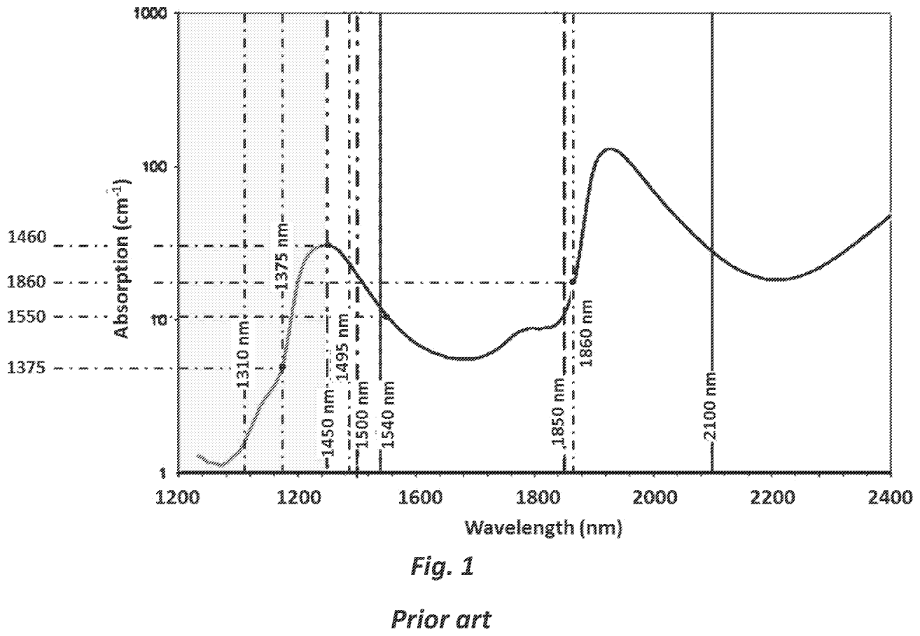

In some embodiments, the wavelength of the generated optical signals is at a range of 1300-1460 nm. Referring back to FIG. 1, as can clearly be seen from the graph, the absorption coefficient of radiant energy in water at wavelengths between 1300-1460 nm, is low relatively to wavelengths between 1870-1950 nm, and additionally have larger scattering and thus was considered unsuitable for nerve stimulation. However, since this range is within the telecommunications range thus benefits from lower cost, reliable, readily available and most importantly efficient equipment. Therefore, an optical system consisting of signal generator 190, delivery element 220 and light emitter 222 for generating, delivering and emitting photon pulses at a wavelength of at most 1600 nm, for example, 1300-1460 nm may be significantly more efficient for stimulation (e.g., consume less energy) and at significantly lower cost than similar system used for generating, delivering and emitting photons at wavelength longer than 1700 nm.

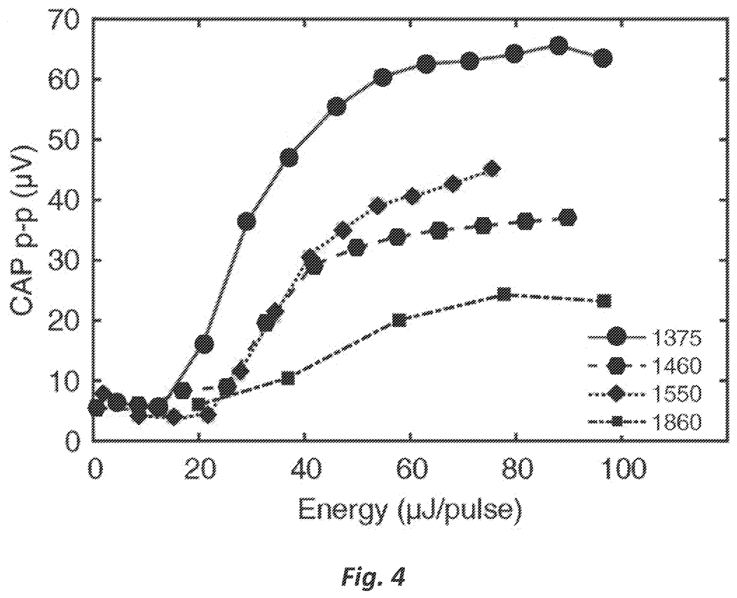

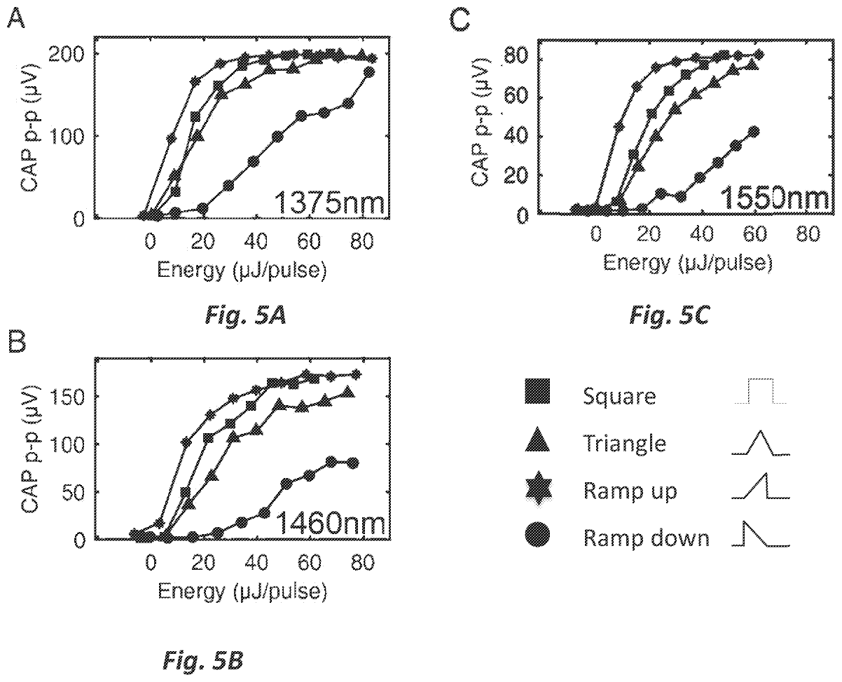

INS at shorter wavelengths evoked CAPs with larger amplitude than radiation at the commonly used wavelength (1800-2100 nm). The CAP is the electrical voltage recorded extracellularly from population of nerves during nerve stimulation. The CAP amplitude (in μV) increase as a function of the radiant energy (in μJ) delivered with each pulse at 4 different wavelengths is given in FIG. 4. The CAP amplitude was measured for different radiation wavelengths in guinea pigs implanted with a CI according to some embodiments of the invention. Similarly, the results shown in FIGS. 5-7 were also received from similar experiments conducted with guinea pigs.

As shown in the graphs of FIG. 4, the lowest CAP amplitude for provided radiant energy was received when the pulse was at a wavelength of 1860 nm and the highest was when the pulse was at a wavelength of 1375 nm. Accordingly, the inventors found that despite the higher energy absorption coefficient at 1860 nm compared to 1375 nm and 1550 nm (e.g., as can be seen in FIG. 1) the stimulation efficiency is higher at wavelengths with a lower absorption coefficient which might be explained by a deeper penetration of the radiation into the nerve tissue resulting in a larger number of stimulated nerves contributing to a larger CAP amplitude. It has also been found that pulsed radiation at wavelengths with longest penetration depth has the lowest thresholds for stimulation and produces the largest CAP response.

In some embodiments, sound processor 110 may be a controller configured to control the generation of the optical signal from optical signal generator 190. Sound processor 110 may send instructions to receiver 180 via transmitter 130. In some embodiments, CI 10 may include a first optical signal generator configured to generate optical signals at a first wavelength and a second optical signal generator configured to generate optical signals at a second wavelength. In some embodiments, the controller (e.g., sound processor 110) may be configured to control the first optical signal generator to generate a first portion of the plurality of optical signals and control the second optical signal generator to generate a second portion of the plurality of optical signals.

For example, photon generation source 194 may include two or more different types of laser diodes or LEDs each configured to generate photons at a different wavelength, for example, 1470 nm and 1375 nm. In some embodiments, sound processor 110 may be configured to control a first laser diode to emit light at 1470 nm, for example, to stimulate a small group of nerves resulting in a small CAP to mimic gentle voices and to control a second laser diode to emit light at 1375 nm to stimulate larger number of nerves resulting in a higher CAP to mimic loud voices.

In some embodiments, sound processor 110 may further be configured to receive a captured acoustical signal and divide the acoustical signal into a plurality of frequency bands. In some embodiments, sound processor 110 may assign each frequency band with a specific light emitter (e.g., light emitters 222) corresponding to the location of each light emitter in the cochlea. In some embodiments, sound processor 110 may control the at least one optical signal generator to deliver at least one optical signal to each one of the assigned light emitters according the acoustical signal.

In some embodiments, in order to increase the efficiency of the stimulation (e.g., increase the CAP amplitude for provided radiant energy) the shape of the provided signal or pulse may be altered. In some embodiments, sound processor 110 may be configured to control the at least one optical signal generator (e.g., optical generator 190) to generate the plurality of optical signals at a selected pulse shape, selected from: a square shaped pulse, a ramp up shaped pulse, a ramp down shaped pulse, a triangular shaped pulse, or an exponentially rising pulse.

In some embodiments, sound processor 110 may be configured to select the wavelength, the radiant energy delivered, and the pulse shape. The combined effect of these three parameters is shown in the traces of FIGS. 5A-5C. FIG. 5A shows the traces of the CAP amplitude vs. pulse energy for different pulse shapes delivered at 1375 nm. FIG. 5B shows traces of the CAP amplitude vs. radiant energy for different pulse shapes delivered at 1460 nm. FIG. 5C shows the traces of the CAP amplitude vs. pulse energy for different pulse shapes delivered at 1550 nm. As shown, the most efficient combination for radiation wavelength and pulse shape is delivering pulses at 1375 nm by a ramp-up shaped pulse. The ramp-up shape was the most efficient way to stimulate nerves (e.g., generated the highest CAP amplitude) at every tested wavelength. The ramp-down was the most inefficient pulse shape.

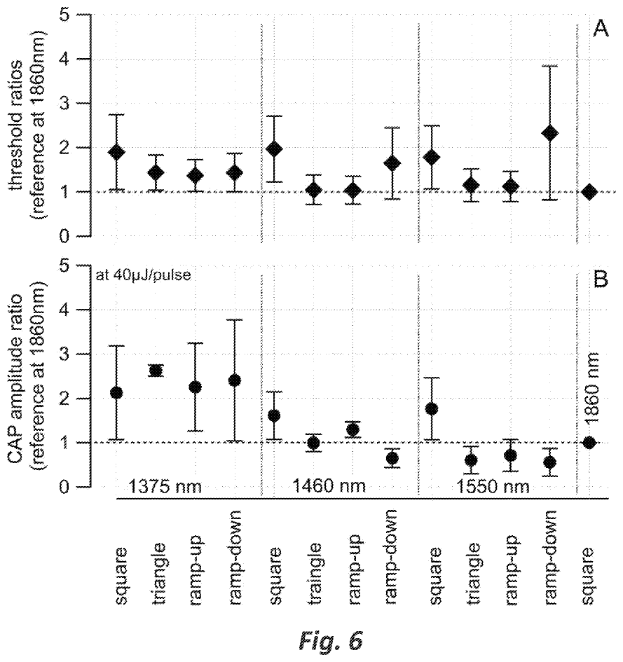

Reference is now made to FIG. 6, which shows the CAP amplitude and the threshold radiant energy for stimulation at 3 different wavelengths and various pulse shapes, relative to the values achieved with square shaped pulses at 1860 nm (typically used in previous research on cochlear optical stimulation) according to some embodiments of the invention. The experiments were conducted in a population study of 11 animals. The best results were achieved when the stimulation was provided at 1375 nm. Evaluation of the results with a linear mixed-effect model of wavelength and pulse shape, show that the wavelength plays a larger role than the pulse shape.

To the surprise of the inventors, the disclosed shorter wavelengths which were known in the art to have relatively lower radiant energy absorption in the target tissue and higher scattering, and were not expected to efficiently stimulate the cochlear nerve, as disclosed herein above, resulted in a more efficient nerve stimulation than the known in the art longer (e.g., higher than 1800 nm) wavelengths.

In addition, the optical devices that generate and deliver these shorter wavelengths are known to have much better wall-plug-efficiency.

Accordingly, a CI according to some embodiments of the invention may have higher energy efficiency, higher reliability and may generate higher CAP at much lower costs than all the optical CIs known in the art.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A cochlear implant, comprising:
at least one optical signal generator configured to generate a plurality of optical signals having a wavelength of at most 1600 nm; and a plurality of light emitters, for delivering the optical signals to different locations along a cochlear nerve.

2. The cochlear implant of claim 1, wherein at least one optical signal generator is configured to generate the signals at a wavelength of 1300-1460 nm.

3. The cochlear implant of claim 1, wherein the plurality of light emitters are one or more optical projection elements selected from a group consisting of: optical gratings, lenses, mirrors and prisms and the cochlear implant further comprising at least one waveguide for delivering the generated optical signals from the at least one optical signal generator to the plurality of light emitters.

4. The cochlear implant of claim 3, wherein the at least one waveguide is made from optical polymeric material which is shaped to fit a cochlea.

5. The cochlear implant of claim 4, wherein each waveguide is shaped to fit a cochlea of a specific patient.

6. The cochlear implant of claim 3, comprising a bundle of waveguides and wherein the bundle of waveguides is shaped to fit a cochlea of a specific patient.

7. The cochlear implant according to claim 3, wherein the at least one waveguide comprises one or more optical amplifiers, embedded in the at least one waveguide for amplifying at least some of the plurality of optical signals.

8. The cochlear implant according to claim 3, wherein the at least one optical signal generator is a photon generating source selected from, laser diode and light emitting diodes (LEDs).

9. The cochlear implant of claim 1, wherein the optical signal generator is an electrical power source and the plurality of light emitters are selected from a group consisting: laser diodes and LEDs and the cochlear implant further comprising at least two wires configured to provide electricity to the laser diodes or the LEDs.

10. The cochlear implant according to claim 1, further comprising:
a receiver configured to receive instructions from a controller, wherein the controller is configured to:
control the at least one optical signal generator to generate the plurality of optical signals.

11. The cochlear implant of claim 10, wherein the controller is further configured to:
control the at least one optical signal generator to generate the plurality of optical signals at a selected pulse shape, selected from: a square shaped pulse, a ramp up shaped pulse, a ramp down shaped pulse, a triangular shaped pulse and exponentially rising pulse.

12. The cochlear implant of claim 10, wherein the controller is further configured to:
control the at least one optical signal generator to generate the plurality of optical signals at two or more different wavelengths.

13. The cochlear implant of claim 12, comprising:
a first optical signal generator configured to generate optical signals at a first wavelength; and
a second optical signal generator configured to generate optical signals at a second wavelength, and wherein the controller is further configured to:
control the first optical signal generator to generate a first portion of the plurality of optical signals; and
control the second optical signal generator to generate a second portion of the plurality of optical signals.

14. The cochlear implant according to claim 10, wherein the controller is further configured to:
receive a captured acoustical signal;
divide the acoustical signal into a plurality of frequency bands;
assign each frequency band with a specific light emitter; and
control the at least one optical signal generator to deliver at least one optical signal to each one of the assigned light emitters according the acoustical signal.

15. A method of generating stimulations for a cochlear implant, comprising:
generating, by an optical signal generator, a plurality of optical signals having a wavelength of at most 1600 nm; and
delivering the generated plurality of optical signals at the one or more locations in a cochlea using one or more light emitters.

16. The method of claim 15, wherein a wavelength of the generated optical signals is at a range of at 1300-1460 nm.

17. The method of claim 15, wherein the optical stimulations are generated at a selected pulse shape, selected from: a square shaped pulse, a ramp up shaped pulse, a ramp down shaped pulse, a triangular shaped pulse and exponentially rising pulse.

18. The method according to claim 15, wherein generating the plurality of optical stimulations is in two different wavelengths.

19. The method of claim 18, wherein a first wavelength is selected to penetrate to a first tissue penetration depth and the second wavelength is selected to penetrate to a second tissue penetration depth, deeper than the first tissue penetration depth.

20. The method according to claim 15, further comprising:
amplifying at least some of the plurality of optical signals using one or more optical amplifiers, embedded in at least one waveguide for delivering the plurality of optical signals to the one or more light emitters.

21. The method according to claim 15, further comprising:
capturing an acoustical signal;
dividing the acoustical signal into a plurality of frequency bands;
assigning each frequency band with a specific light emitter; and
delivering at least one optical signals to each one of the assigned light emitters according to an acoustical signal.
